(19) United States
(12) Patent Application Publication
(10) Pub. No.: US 2009/0287156 A1
(43) Pub. Date: Nov. 19, 2009

(54) Fixture for Mounting a Gastrostomy Tube on a Base, and Gastrostomy Button

(76) Inventor: Stephane Regnault, Rueil-Malmaison (FR)

Correspondence Address: LEVINE & MANDELBAUM, 222 Bloomingdale Road, Suite 203, WHITE PLAINS, NY, 10605, US

(21) Appl. No.: 12/307,151

(22) PCT Filed: Jul. 6, 2007
(86) PCT No.: PCT/EP07/56914

§ 371 Date: Jan. 15, 2009

(30) Foreign Application Priority Data
Jul. 6, 2006 (FR) 0606140

(51) Int. Cl. A61M 5/32

(52) U.S. Cl. 604/174

(57) ABSTRACT

Fixture for mounting a gastrostomy tube (15) on a base to be applied to the skin around a stoma, said base comprising a connector piece (22) for mounting the tube on the base, characterized in that the connector piece has an external thread (23), and in that the tube and the connector piece are chosen such that the tube, cut to the required length, can be engaged with force on the connector piece and turned about itself in such a way that the thread of the connector piece imprints itself in the wall of the tube, thereby creating corresponding helical lines that ensure considerable resistance to traction between the tube and the connector piece.

A gastrostomy tube is designed to be inserted in a passage (stoma) which passes through the skin and abdominal wall until it opens into the gastric cavity.

Said tube is normally fixed on a base applied on the skin around the stoma, said base defining a transit chamber with which the tube must communicate.

The present invention relates to the fixture of the gastrostomy tube on the base.

Said fixture must meet the following conditions:
enable adaptation of the length of the tube to the length of the stoma,
be compact in size,
provide a satisfactory elasticity between the base, the tube and the stoma,
be removable.

Solutions are known consisting of selecting the tube from a collection of tubes of different lengths, as provided for example in the publications U.S. Pat. No. 4,863,438 and U.S. Pat. No. 5,336,203. This type of solution makes it possible to shape the proximal end of the tube specially so as to ensure the fixture on the base and tightness but it involves having a collection of tubes of different lengths and only allows an approximate adaptation of the length of the tube to that of the stoma.

Solutions are also known wherein the tube is cut to the required length to adapt the length thereof, as provide for example in the publication U.S. Pat. No. 4,944,732. This type of solution enables a precise adaptation of the length of the tube but requires having a high-performance solution to fix the tube on the base tightly.

A known solution, as described in the publication U.S. Pat. No. 4,944,732, consists of engaging and gluing the end of the tube onto a tubular connector piece of the base.

This solution does not allow a disassembly of the tube and does not guarantee a sufficient resistance to traction between the tube and the connector piece.

The present invention is intended to make it possible to cut the tube on demand to adapt same to the length of the stoma, avoiding the above-mentioned drawbacks.

This is achieved, according to the invention, by fitting the base with an externally threaded tubular connector piece, and by selecting the tube and the connector piece such that the tube, cut to the required length, can be engaged with force on the connector piece and turned about itself in such a way that the threading of the connector imprints itself on the wall of the tube, thereby creating the corresponding helical lines.

The materials of the tube and the connector pieces and the transversal dimensions of the tube and the connector piece are selected accordingly.

This fixture ensures considerable resistance to traction between the tube and the connector piece.

If the patient decided to pull strongly on the base, a simple tube fixture on a smooth connector piece would not be sufficient, as slippage would occur. On the other hand, the threads/counter-threads resist said traction mechanically without slipping. Moreover, when the tube is inserted into the stomach, if the base is turned, the tube also turns in the same directions, rendering unscrewing impossible.

In a preferred solution, the connector piece is part of a metallic insert incorporated in the base.

Advantageously, said insert is designed to carry out a triple function:
mounting of the tube
housing of the valve
holding of the cap.

The device may be completed by a washer made of an elastic plastic material previously placed on the tube adjusted in length before “screwing” the tube on the threaded connector piece of the insert.

When the tube is screwed onto the connector piece, said washer is pushed against the lower face of the base. The washer will clamp the tube against the threads of the connector piece and improve the resistance to traction of the tube on the base.

As an example, an embodiment according to the invention will be described hereinafter with reference to the figures in the drawing attached wherein:

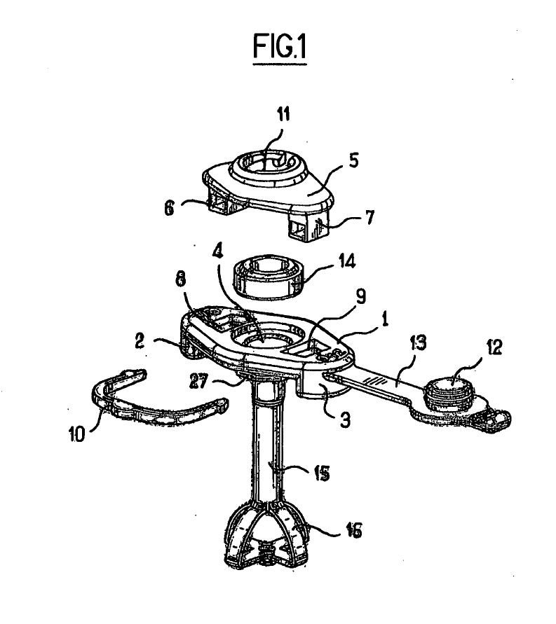

FIG. 1 is an exploded schematic perspective view of a gastrostomy button;

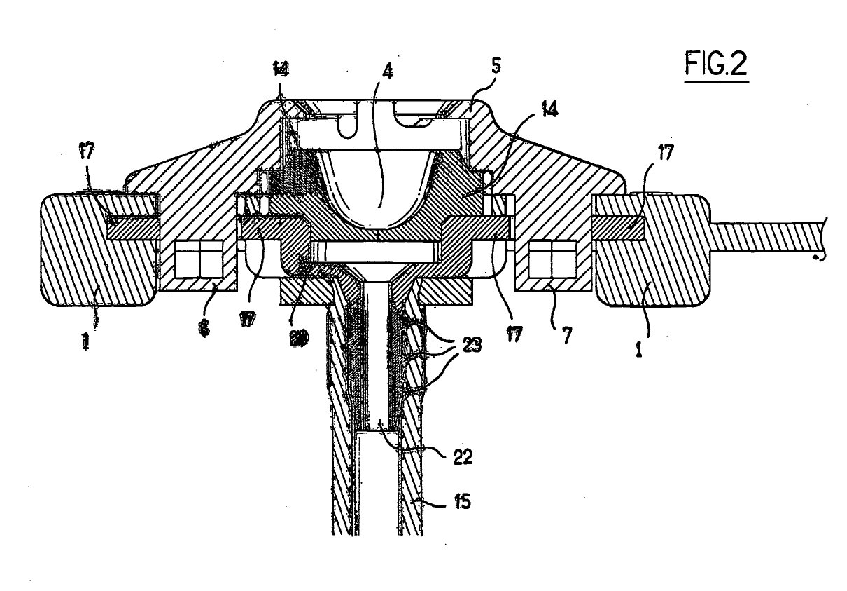

FIG. 2 is an axial cross-section of the button in FIG. 1, after assembly;

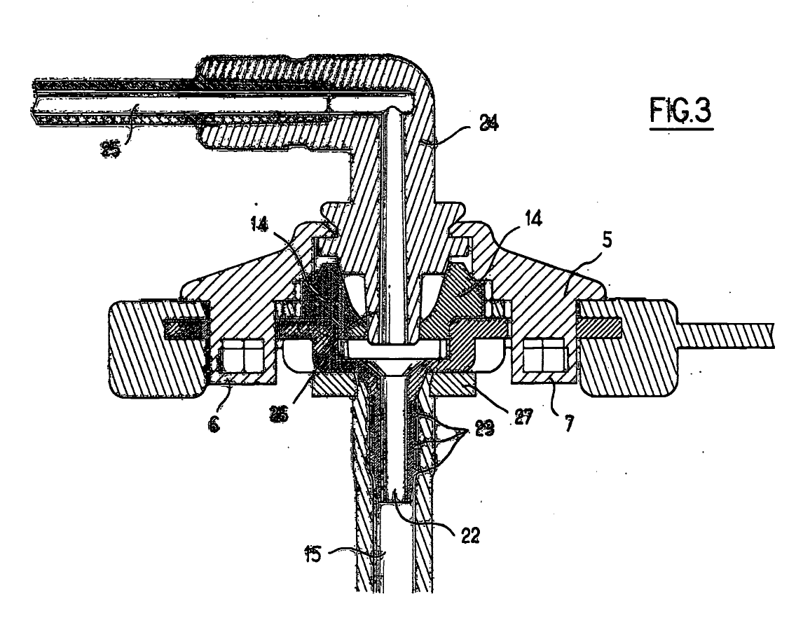

FIG. 3 is similar to FIG. 2 but with a connector connected to the base;

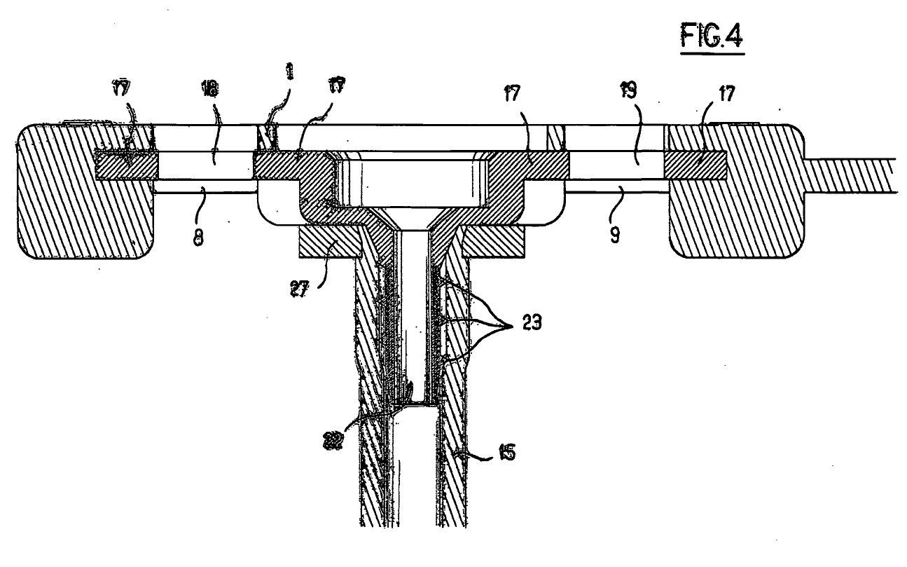

FIG. 4 is an axial cross-section of the base, the cap being removed;

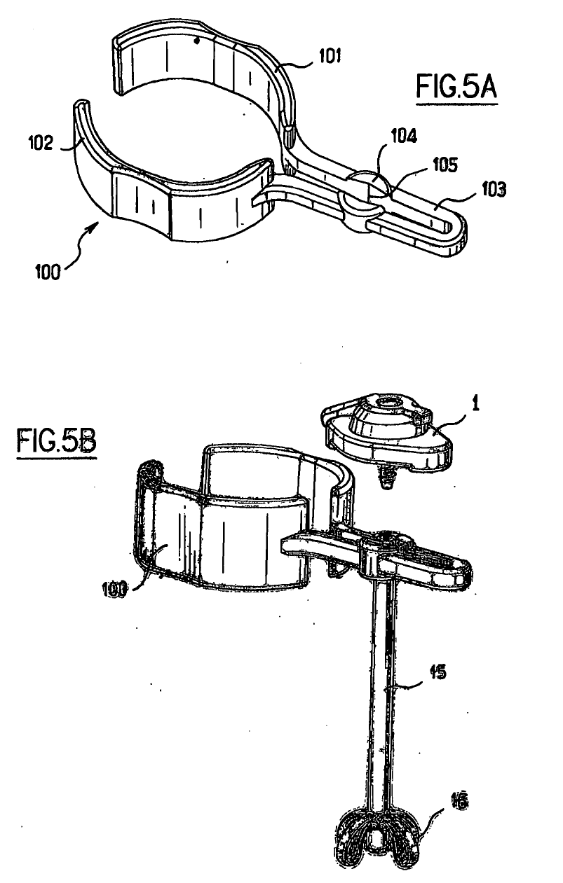

FIG. 5A is a three-dimensional view of a mounting instrument of the base on the gastrostomy tube to form a gastrostomy button according to the invention;

FIG. 5B is a three-dimensional view of the fixture of a gastrostomy button with the instrument in FIG. 5A;

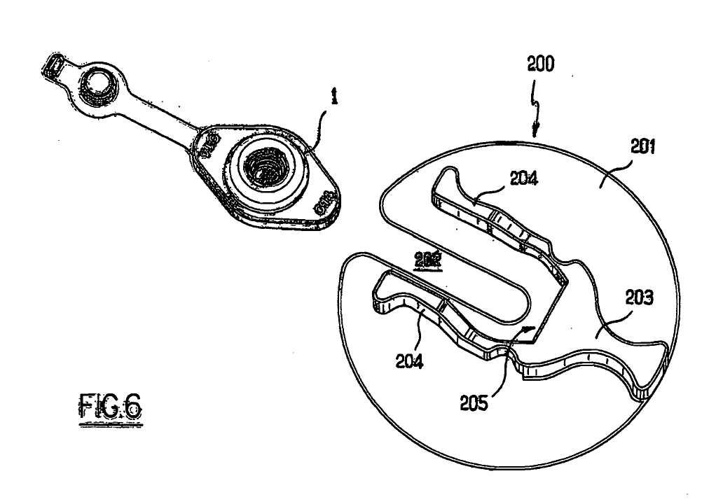

FIG. 6 is a three-dimensional view of a securing instrument of a gastrostomy button according to the invention;

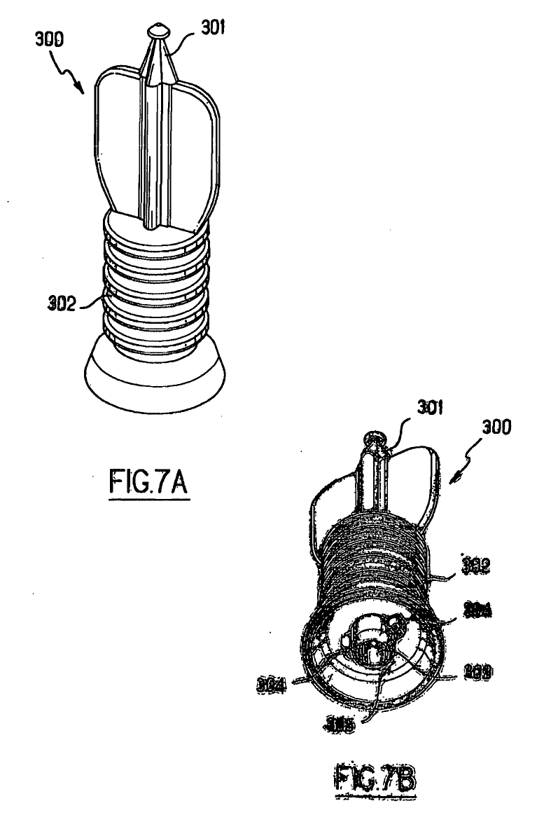

FIGS. 7A and 7B are three-dimensional views of a valve change instrument of a gastrostomy button according to the invention;

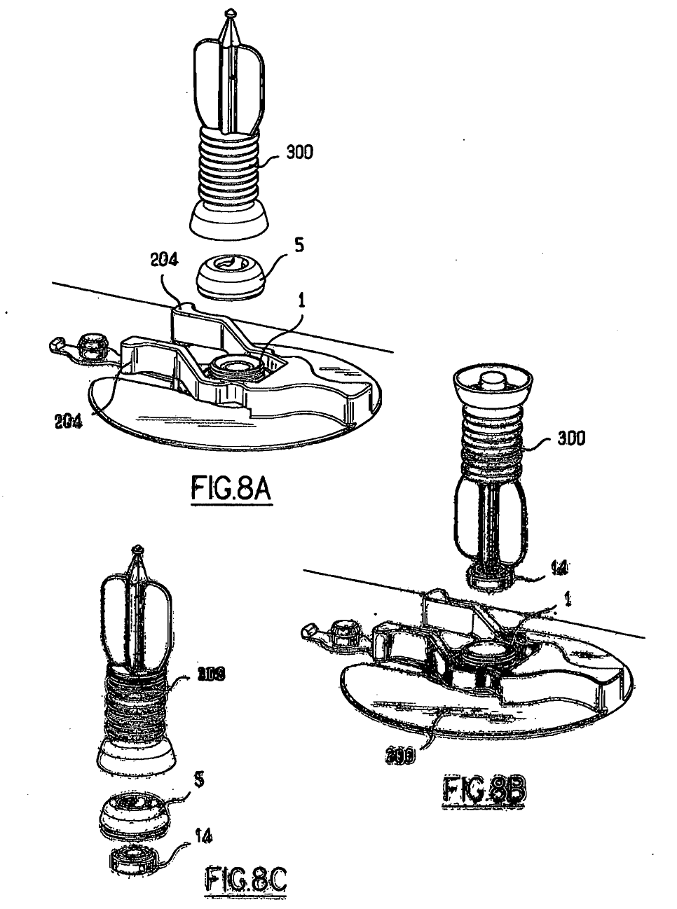

FIGS. 8A to 8C are three-dimensional views of the valve changing steps of a gastrostomy button according to the invention;

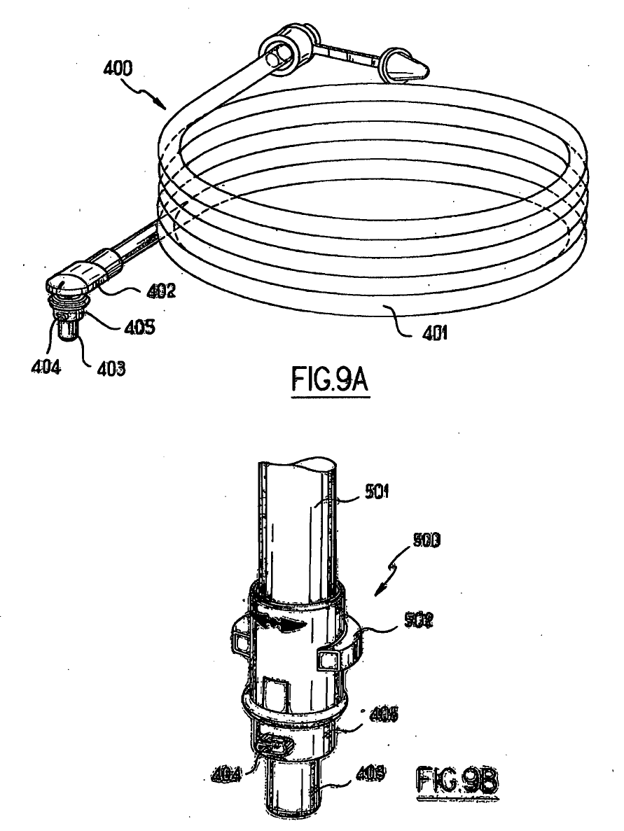

FIG. 9A is a three-dimensional view of a feed extension equipped with a curved connector piece that can be connected to the gastrostomy button according to the invention, and;

FIG. 9B is a three-dimensional view of a straight connector piece for the feed extension in FIG. 9A.

In FIG. 1, a gastrostomy “button” consisting of the following parts has been represented:
a base 1 made of synthetic resin to be applied on the skin around a stoma, said base comprising two application supports 2,3 and determining a transit chamber 4 passing through the base in the axis of the stoma;
a cap 5 fixed on the base at the top of the chamber 4, by inserting two tabs 6,7 located on the under-face of the cap in two corresponding passages 8,9 formed via the base and securing said tabs by means of a clip 10 inserted laterally under the base between the base supports. Said cap determines an open passage 11 communicating with the chamber;
a cover 12 attached to the base by a tab 13 and folding back onto the cap to close the cap passage;
a valve 14 to be inserted into the chamber 4 of the base;
a tube 15 made of synthetic resin cut to the required length, mounted on the base in the axis of the chamber, said tube being intended to pass through the stoma and be retained in the gastric cavity by a securing element 16.

Said button defined in this way displays specific advantages over buttons according to the prior art but does not represent a non-limitative example of a button to which the present invention can be applied.

In fact, the invention firstly relates to the fixture of the tube 15 on the base 1.

According to the invention (FIG. 2), the base 1 comprises a metallic insert shaped to form a disk 17 embedded in the base, drilled with two lateral holes 18,19 for the passage of the tabs of the cover, a hub 20 formed at the centre of the disk to be housed in the chamber 4 by determining a central hollow 21 suitable for receiving in part the valve 14 and a tubular connector piece 22 located in the axis of the hub, communicating with the chamber and protruding under the base.

The metallic connector piece 22 displays an external thread consisting of fine, sharp-edged threads 23.

The materials and the diameters of the proximal end of the tube and the connector piece are selected such that the tube 15 can be engaged with force on the connector piece and turned about itself in such a way that the threading 23 of the connector imprints itself on the internal face of the wall of the tube.

The fixture of the tube 15 on the base 1 is reinforced by an elastic washer 27.

FIG. 3 is a view corresponding to that of FIG. 2 but with a curved connector 24 inserted into the passage 11 of the cap to connect said passage and the chamber of the base with a feed catheter 25.

Said connector 24 comprises a connector piece 26 which fits with force via the valve 14 with lateral tightness.

According to an alternative embodiment of the base 1, the cap 5 is screw-mounted on the base on top of the chamber 4.

With reference to FIGS. 5A and 5B, an instrument 100 for the mounting of the base 1 on the gastrostomy tube 15 will be described hereinafter. The instrument 100 is in the form of a clip comprising two handles 101, 102 forming the ends of two arms forming an elastically deformable “U” 103. At a centre of each of the arms of the “U”, the instrument comprises interfacing means 104 with the gastrostomy tube 15. The interfacing means 104 comprise a substantially rectilinear groove 105, substantially perpendicular to the arms of the “U” 103, the groove being open to the inside of the “U”. The groove 105 is suitable for receiving the gastrostomy tube 15.

During mounting of the base 1 on the tube 15, the end opposite the securing element 16 is clamped in the interfacing means 104, the tube being received in the groove 105. The base is then screwed in the tube as described above. The mounting instrument 100 is made of plastic and makes it possible to avoid deforming the tube while limiting the possible clamping. In this way, when the base comes to a stop on the tube at the end of clamping, if the clamping movement continues, the button assembly turns, with the tube 15 turning in the mounting instrument 100. The destruction of the thread produced in the tube is avoided and the satisfactory long-term resistance of the assembly forming the button is ensured.

If, after several months (for example) of use, the button is to be changed, a securing instrument 200 as illustrated in FIG. 6 is used first of all. This instrument 200 comprises a plate 201, of a circular shape, displaying an opening 202 in a tapered “U” shape extending substantially radially from a centre of the plate to a radially external circumference. Said opening is suitable for receiving the gastrostomy tube with sliding. A handle 203 extends in projection from a surface of the plate in the diametral prolongation of the opening 202 and comprises an open cavity 205 to the centre and the opening 202. Said cavity is suitable for receiving one of the supports of the base with sliding. The securing instrument also comprises two elastically deformable wings 204 extending opposite each other at either end of the opening 202. Once deformed by moving them closer to each other, said wings make it possible to hold the base in place in the instrument, on one hand, and, on the other, lock the button in rotation and in detachment or pressure movement on the skin, during use.

With reference to FIGS. 7A and 7B, a valve changing instrument 300 will be described hereinafter. Said instrument is of a general cylindrical revolution shape. It comprises a first end 301 in a harpoon shape. It comprises a second end 303 with a relief shape 303,304 that is substantially complementary to the external shape of the cap 5. The relief shape comprises two diametrically opposed fins 304, extending in the hollow shape. Between the two ends, the valve changing instrument comprises a handle 302.

With reference to FIGS. 8A to 8C, the securing instrument 200 is inserted between the skin and the base 1 and the valve changing instrument 300 is connected in the cap 5 by means of the end 303 thereof. The cap is then unscrewed by engaging the fins 304 engaging with complementary shapes provided in the periphery of the open passage 11, which are of the bayonet type. This makes it possible to remove the cap 5. The worn valve 14 is then “harpooned” with the other end 301 of the valve changing instrument and removed as illustrated in FIG. 8B. The cap 5 is replaced in the hollow shape of the end 303, and the new valve 15 is inserted into the cap 5, and the assembly is fixed again on the base using the valve changing instrument 300.

With reference to FIGS. 9A and 9B, a feed extension 400, 500 suitable for being mounted on a gastrostomy button according to the invention is described hereinafter. Said extension comprises a flexible tube 401, 501 wherein one of the ends comprises a connector piece 402, 502. Said connector piece may be curved 402 or straight 502. In both cases, the connector piece comprises one cylindrical revolution end 403 suitable for operating on the valve 15 so as to open same when the connector piece is connected to the gastrostomy button via the open passage 11. Said end is topped with a part 405 of a complementary cylindrical revolution shape to that of the open passage 11. Two diametrically opposed fins 404 extend in projection from the part 402 and are suitable for cooperating with the complementary bayonet type shapes provided in the periphery of the open passage 11. This makes it possible to make a bayonet connection with locking of the connector piece in the base.

The invention is not limited to the embodiment described above. It is also particularly applicable to any type of base, irrespective of the fixture of the cap on the base and the other embodiment details of the button.

1. Fixture of a gastrostomy tube cut to the required length on a base to be applied on the skin around a stoma, said base comprising a connector piece for mounting the tube on the base, wherein the connector piece comprises an external thread, and in that the tube and the connector piece are chosen such that the tube, cut to the required length, can be engaged with force on the connector piece and turned about itself in such a way that the thread of the connector piece imprints itself in the wall of the tube, thereby creating corresponding helical lines that ensure considerable resistance to traction between the tube and the connector piece.

2. Fixture according to claim 1 wherein the tube comprises a synthetic resin.

3. Fixture according to claim 1 wherein said connector piece is metallic.

4. Fixture according to claim 1 wherein the connector piece of the base is part of an insert embedded in the base.

5. Fixture according to claim 1 further comprising an elastic washer 27 inserted on the tube, before screwing the tube on the connector piece, to clamp the tube against the threads of the connector piece.

6. Gastrostomy button comprising a base made of synthetic resin to be applied on the skin around a stoma, said base determining a transit chamber passing through the base, a cap fixed on the base at the top of the chamber, said cap determining an open passage communicating with the chamber, a cover to close the cap, and a gastrostomy tube cut to the required length and fixed on the base, wherein the base comprises an insert forming an external threaded connector piece protruding under the base and in that the diameters and the materials of the connector piece and the tube are selected such that the tube can be engaged with force and by turning on the connector piece and in that the thread of the connector piece imprints itself in the wall of the tube, thereby creating a corresponding thread in the internal face of the wall of the tube.

7. Button according to claim 6, wherein said insert comprises a disk embedded in the base and a connector piece protruding on one face of said disk.

8. Button according to claim 7 wherein the disk of the insert has a plurality holes for the passage of respective fixture tabs of the cap on the base.

9. Button according to claim 6 wherein the insert comprises a hub formed at the centre of the disk to house a valve in the chamber of the base.

10. Button according to claim 6 further comprising an elastic washer inserted on the tube to clamp the tube against the threads of the connector piece.

11. Button according to claim 6 wherein said insert is metallic and said tube comprises a synthetic resin.
